(19) United States
(12) Patent Application Publication
(10) Pub. No.: US 2003/0204318 A1
(43) Pub. Date: Oct. 30, 2003

(54) Determination of interference RNAs (iRNAs) and small temporal RNAs (stRNAs) and their interaction with connectrons in prokaryotic, archea and eukaryotic genomes

(76) Inventor: Richard J. Feldmann, Derwood, MD (US)

Correspondence Address: Jim Zegeer, Law Office of Jim Zegeer, Suite 108, 801 North Pitt Street, Alexandria, VA, 22314, US

(21) Appl. No.: 10/339,674

(22) Filed: Jan. 10, 2003

Related U.S. Application Data
(63) Continuation-in-part of application No. 10/227,568, filed on Aug. 26, 2002, now abandoned.
Application No. 10/227,568 is a continuation-in-part of application No. 09/866,925, filed on May 30, 2001.
(60) Provisional application No. 60/347,257, filed on Jan. 14, 2002.

(51) Int. Cl. G06F 19/00; G01N 33/48; G01N 33/50

(52) U.S. Cl. 702/20

(57) ABSTRACT

A computational method has been developed to detect the conditions whereby gene expression control mechanisms will stop the transcription of RNA that would otherwise be used to form a connectron.

### REFERENCE TO RELATED APPLICATION

The present application is the subject of Provisional Application Serial No. 60/347,257 filed Jan. 14, 2002

The present application is a continuation in part of U.S. patent application Ser. No. 09/866,925 filed May 30, 2001 entitled ALGORITHMIC DETERMINATION OF FLANKING DNA SEQUENCES THAT CONTROL THE EXPRESSION OF SETS OF GENES IN PROKARYOTIC, ARCHEA AND EUKARYOTIC GENOMES, incorporated herein by reference.

The present application is an continuation in part of U.S. patent application Ser. No. 10/227,568 filed Aug. 26, 2002 entitled Determination of flanking DNA sequences that control the expression of sets of genes in the Escherichia coli K-12 MG1655 complete genome, incorporated herein by reference.

### INTRODUCTION

The connectron structure of a genome determines sets of four DNA sequences of minimum length of 15-bases (C1 and C2 which are in the 3′UTR of a gene, T1 which is on the 5′-side and T2 which is on the 3′-side of a set of genes). When some genes are transcribed into RNA the C1 and C2 sequences in the 3′UTR form the source of a connectron. In addition to binding to the T1 and T2 target sequences, the C1/C2 sequences can also bind to the DNA double-stranded sequences of other equivalent C1/C2 sequences that happen to lie elsewhere in the genome but in particular in the 3′UTR of other genes. When these triple-stranded RNA-DNA-DNA generalized Hoogsteen helices form, the translation of the DNA into RNA is halted and no additional C1/C2 connectron source sequences are produced. The lifetime of this interference RNA (iRNA) is proportional to length of the C1 and C2 sequences. Only the relative lengths of the lifetimes distinguish iRNAs from small temporal RNAs (stRNAs). This invention deals with the relationship between connectrons, iRNAs and stRNAs, as well as a program method for determining the iRNA and stRNA sequences with their associated lifetimes.

### DEFINITIONS

Interference RNA (iRNA)—Any sequence of RNA that can bind to a double-stranded DNA to form a triple-stranded generalized Hoogsteen helix.

Small Temporal RNA (stRNA)—Any sequence of RNA that can bind to a double-stranded DNA to form a triple-stranded generalized Hoogsteen helix.

### PRIOR ART

A recent article in Science magazine (1) described interference RNA (iRNA) as the most important scientific breakthrough of 2002. This article provided a bibliography (references 2 to 15) that gives a good understanding of how scientists view the role of iRNA, stRNA and several other related RNAS (i.e. microRNA and small interfering RNA). None of these references mention the use of our patent pending invention of the tetradic relationship that we call a connectron nor do they mention the use of iRNA and stRNA in relationship to connectrons.

### BRIEF DESCRIPTION OF THE OBJECT OF THE INVENTION

The object of this invention is to provide a computational method that shows how the transcription of RNA that would otherwise be used to form a connectron can be stopped.

### DESCRIPTION OF THE DRAWINGS

The above and other objects, advantages and features of the invention will become more apparent when considered with the following specification and accompanying drawings and table wherein:

FIG. 1 illustrates (a) Transcription and Editing. (b) Movement of the RNA through the Nucleus. (c) Connectron Formation. (d) Action of the DICER enzyme. (e) Binding of iRNA to double-stranded DNA of C1 and C2 sequences,

FIG. 2 illustrates the overall layout of computer and program,

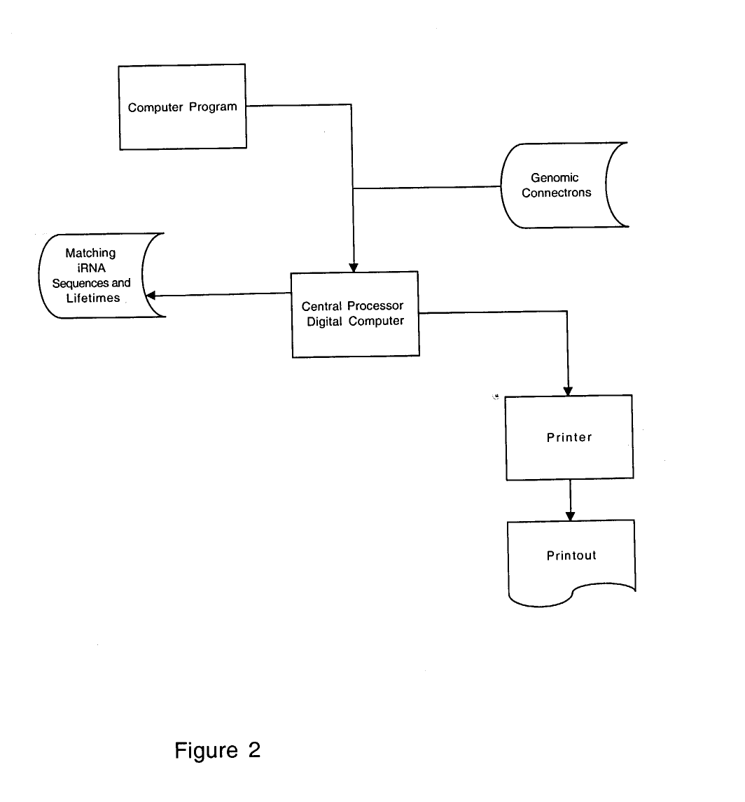

FIG. 3 illustrates the process flow of computer program,

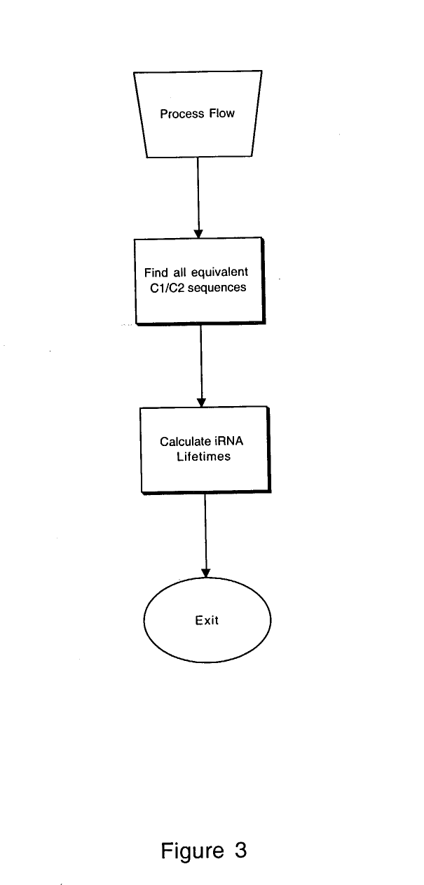

FIG. 4 illustrates the determination of all C1/C2 matches and

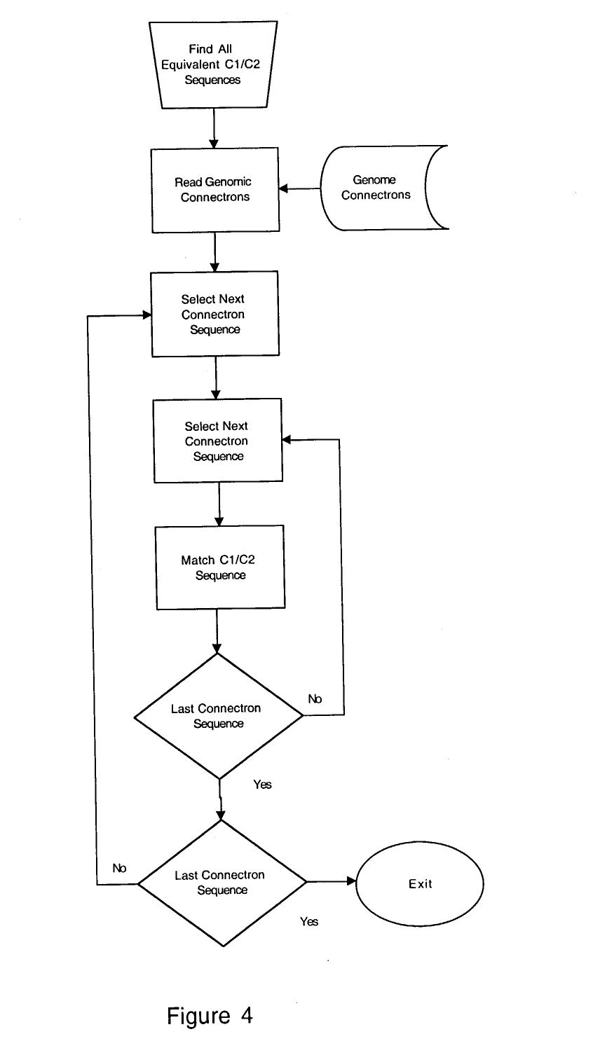

FIG. 5 illustrates the calculation of iRNA lifetimes.

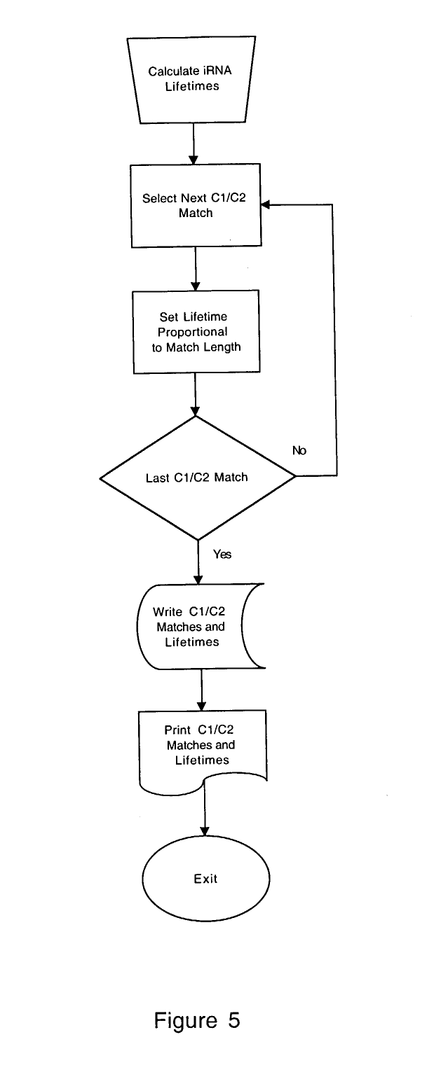

### DESCRIPTION OF THE INVENTION

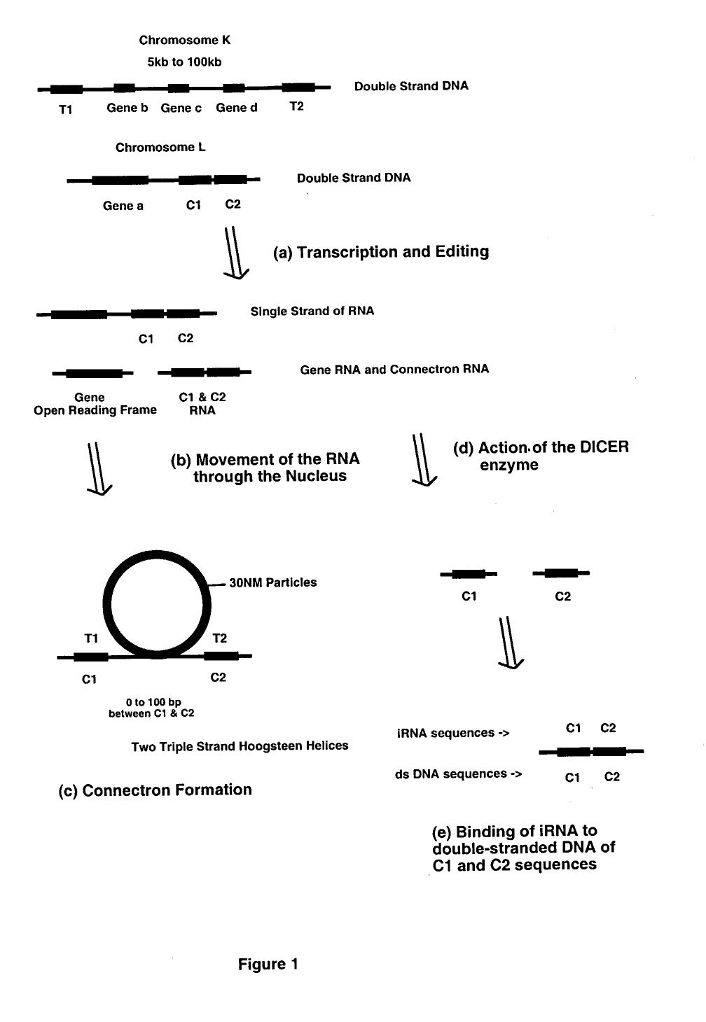

As shown in FIG. 1, single-stranded RNA is produced when a gene is transcribed. The RNA transcript performs three roles. In role one, one or more copies of the RNA transcript may be edited to form the open reading frame mRNA for translation into protein. In role two, the single-stranded RNA can be used for connectron formation. In role three, other copies of the single-stranded RNA are cut into small fragment by the DICER enzyme. Characteristically the DICER enzyme cuts RNA into 21-base fragments. Two of these fragments are the C1 and the C2 sequences. These single-stranded RNA fragments then bind to the respective double-strand cognate DNA sequences to form two short triple-strand generalized Hoogsteen helices. The double-strand DNA sequences of C1 and C2 that are relevant are those that are in the 3′UTR of one or more genes. When the polymerase that is transcribing the double-stranded DNA into RNA comes to the C1 and C2 sequences that have the iRNA bound to them, the polymerase stops its transcribing action. The two generalized Hoogsteen helices act as a block to the formation of more single-stranded RNA of the C1 and C2 sequences. The Hoogsteen helices of both connectrons and iRNA have lifetimes that vary directly with the length of the generalized Hoogsteen helix. The effect of the iRNA (generalized) Hoogsteen helices is to prevent the formation of more C1-C2 RNA during the lifetime of these helices. The total systematic effect is that the first gene to express a particulate C1-C2 sequence inhibits all other genes with the same sequence from generating more C1-C2 sequenced RNA.

This invention provides capabilities that are utilized in our application Ser. No. ______ filed contemporaneously herewith and entitles “Simulation of gene expression control using connectrons, interference RNAs (iRNAs) and small temporal RNAs (stRNAs) in prokaryotic, archea and eukaryotic genomes”. The iRNAs and stRNAs play a vital role in determining the simulation of cellular dynamics. This invention provides a way of utilizing iRNAs and stRNAs within the methodology of connectron control of gene expression.

### EXAMPLE

Connectron 350 is an example of a transient connectron. It is described in E. coli genomic patent application identified above as

| | | C1/C2 | | | T1-T2 | | |
|---|---|---|---|---|---|---|---|
| Global_Id | Chromosome | Cl_Id | C2_Id | Chromosome | T1_Id | T2_Id | Connectron_Type |
| 350 | 1 | 26 | 26 | 1 | 321 | 346 | transient |

The C1/C2 source of the transient connectron 350 is represented in as

| Type | Num | Jobno | Chr | Start | Stop | Length | GeneName |
|---|---|---|---|---|---|---|---|
| CNT | 26 | 1 | 1 | 19.796 | 19.859 | .064 | --> \| \| \| \| \| \| \| \| \| \| \| \| \| \| |

The “Type” descriptor of this transient C1/C2 connectron source is “CNT”. The letter “N” indicates that the C1/C2 connectron source occurs on the negative strand of the double-stranded DNA of the genome. The letter “P” in this place would indicate a C1/C2 connectron source on the positive strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB).

Connectron 19340 is an example of a transient connectron. It is described in E. coli genomic patent application identified above as

| | | C1/C2 | | | T1-T2 | | |
|---|---|---|---|---|---|---|---|
| Global_Id | Chromosome | Cl_Id | C2_Id | Chromosome | T1_Id | T2_Id | Connectron_Type |
| 19340 | 1 | 1260 | 1260 | 1 | 321 | 346 | transient |

The C1/C2 source of the transient connectron 19340 is represented in as

| Type | Num | Jobno | Chr | Start | Stop | Length | GeneName |
|---|---|---|---|---|---|---|---|
| CPT | 1260 | 1 | 1 | 1049.705 | 1049.769 | .065 | --> \| \| \|\|\|\| \|\|\|\|\|\|\|\|\|\|\|\|\|\|\|\| |

Connectron 23879 is an example of a transient connectron. It is described in E. coli genomic patent application identified above as

| | | C1/C2 | | | T1-T2 | | |
|---|---|---|---|---|---|---|---|
| Global_Id | Chromosome | Cl_Id | C2_Id | Chromosome | T1_Id | T2_Id | Connectron_Type |
| 23879 | 1 | 1927 | 1927 | 1 | 321 | 346 | transient |

The C1/C2 source of the transient connectron 23879 is represented in as

| Type | Num | Jobno | Chr | Start | Stop | Length | GeneName |
|---|---|---|---|---|---|---|---|
| CPT | 1927 | 1 | 1 | 1976.526 | 1976.590 | .065 | --> \|\| \|\|\|\|\|\|\|\|\|\|\|\|\|\|\|\|\|\|\|\|\| |

Connectron 45018 is an example of a transient connectron. It is described in E. coli genomic patent application identified above as

| | | C1/C2 | | | T1-T2 | | |
|---|---|---|---|---|---|---|---|
| Global_Id | Chromosome | Cl_Id | C2_Id | Chromosome | T1_Id | T2_Id | Connectron_Type |
| 45018 | 1 | 3424 | 3424 | 1 | 321 | 346 | transient |

The C1/C2 source of the transient connectron 45018 is represented in as

| Type | Num | Jobno | Chr | Start | Stop | Length | GeneName |
|---|---|---|---|---|---|---|---|
| CPT | 3424 | 1 | 1 | 3581.763 | 3581.827 | .065 | --> \|\| \| \| \|\|\|\| \|\|\|\|\|\|\|\|\|\|\| |

These four connectrons are driven by four C1/C2 instances that share the same 64-base sequence as shown in bold below.

| | | |
|---|---|---|
| C1/C2 | 26 | GCATGACAAAGTCATCGGGCATTATCTGAACATAAAACACTATCAATAAGTTGGAGTCATTACC |
| C1/C2 | 1260 | GCATGACAAAGTCATCGGGCATTATCTGAACATAAAACACTATCAATAAGTTGGAGTCATTACCG |
| C1/C2 | 1927 | GCATGACAAAGTCATCGGGCATTATCTGAACATAAAACACTATCAATAAGTTGGAGTCATTACCC |
| C1/C2 | 3424 | GCATGACAAAGTCATCGGGCATTATCTGAACATAAAACACTATCAATAAGTTGGAGTCATTACCG |

All of the data for the transient connectron 350 are pulled together in the following table that is the “terse” description of the connectron.

Connectron Relationships

| Global_Id | Type |
|---|---|
| 350 | transient |

Control Sequences

| Direction | Chromosome | C1/C2_Id | Start | Stop | Length |
|---|---|---|---|---|---|
| negative | 1 | 26 | 19.859 | 19.796 | .064 |

Trigger Gene

| Name | COG_Id | Start | Stop | Length |
|---|---|---|---|---|
| insb_1 | COG1662 | .508 | 19.811 | .698 |

Target Sequences

| Direction | Chromosome | T1_Id | Start | Stop | Length |
|---|---|---|---|---|---|
| negative | 1 | 321 | 279.118 | 278.386 | .733 |

| T2_Id | Start | Stop | Length |
|---|---|---|---|
| 346 | 290.589 | 289.833 | .757 |

Controlled Genes

| Local_Id | Chromosome | Group | Name | COG_Id | Direction | Start | Stop | Length |
|---|---|---|---|---|---|---|---|---|
| 1 | 1 | Group0058 | insb_2 | COG1662 | positive | 278.402 | 279.099 | .698 |
| 2 | 1 | Group0059 | yagb | — | positive | 279.609 | 281.207 | 1.598 |
| 3 | 1 | Group0059 | yaga | COG1425 | negative | 281.207 | 280.053 | 1.155 |
| 4 | 1 | Group0060 | yage | COG0329 | positive | 281.481 | 284.392 | 2.911 |
| 5 | 1 | Group0060 | yagf | COG0129 | positive | 282.425 | 284.392 | 1.968 |
| 6 | 1 | Group0061 | yagg | COG2211 | positive | 284.619 | 287.623 | 3.004 |
| 7 | 1 | Group0061 | yagh | — | positive | 286.013 | 287.623 | 1.611 |
| 8 | 1 | Group0062 | yagf | COG1414 | positive | 287.628 | 289.529 | 1.901 |
| 9 | 1 | Group0062 | argf | COG0078 | negative | 289.529 | 288.525 | 1.005 |

Controlled Connectrons

| Local_Id | Chromosome | C1/C2_Id | Direction | Start | Stop | Length |
|---|---|---|---|---|---|---|
| 1 | 1 | 327 | negative | 279.335 | 279.136 | .200 |
| 2 | 1 | 337 | negative | 287.273 | 287.259 | .015 |
| 3 | 1 | 339 | negative | 287.296 | 287.282 | .015 |
| 4 | 1 | 342 | negative | 288.502 | 288.471 | .032 |
| 5 | 1 | 345 | negative | 290.589 | 289.833 | .757 |

When gene insb (COG1662) is transcribed, the C1/C2 sequence is produced in the 3′UTR. Depending on how the DICER enzyme works there can be many different fragments. A few such fragments are shown below

First example of a DICER cut of C1/C2 26

GCATGACAAAGTCATCGGGCA TTATCTGAACATAAAACAC TATCAATAAGTTGGAGTCATT

Second example of a DICER cut of C1/C2 26

CATGACAAAGTCATCGGGCAT TATCTGAACATAAAACACT ATCAATAAGTTGGAGTCATTA

Third example of a DICER cut of C1/C2 26

ATGACAAAGTCATCGGGCATT ATCTGAACATAAAACACTA TCAATAAGTTGGAGTCATTAC

A given operation of the DICER enzyme will produce one of these examples or similar examples. The iRNA fragments will then bind as triple-stranded helices to the equivalent sequences in the C1/C2 instances 1260, 1927, and 3424. When the genes associated with these C1/C2 sequences transcribe, the polymerase will find the sequence instances 1260, 1927 and 3424 blocked by triple-stranded generalized Hoogsteen helices formed by the iRNA from C1/C2 26.

### REFERENCES

(1) J. Couzin, “Small RNAs Make Big Splash,” Science 297,2296 (2002)

(2) I. M. Hall et al., “Establishment and Maintenance of a Heterochromatin Domain,” Science 297, 2232 (2002)

(3) K. Mochizuki et al., “Analysis of a piwi-Related Gene Implicates Small RNAs in Genome Rearrangement in Tetrahymena,” Cell 110, 689 (2002)

(4) S. D. Taverna et al., “Methylation of Histone H3 at Lysine 9 Targets Programmed DNA Elimination in Tetrahymena,” Cell 110, 701 (2002)

(5) B. J. Reinhart and D. P. Bartel, “Small RNAs Correspond to Centromere Heterochromatic Repeats,” Science 297, 1831 (2002)

(6) T. A. Volpe et al., “Regulation of Heterochromatic Silencing and Histone H3 Lysine-9 Methylation by RNAi,” Science 297, 1833 (2002)

(7) S. M. Elbashir et al., “Duplexes of 21-Nucleotide RNAs Mediate RNA Interference in Cultured Mammalian Cells,” Nature 411, 494 (2001)

(8) M. Lagos-Quintana et al., “Identification of Novel Genes Coding for Small Expressed RNAs,” Science 294, 853 (2001)

(9) N. C. Lau et al., “An Abundant Class of Tiny RNAs with Probable Regulatory Roles in Caenorhabditis elegans,” Science 294, 858 (2001)

(10) Rosalind C. Lee and Victor Ambros, “An Extensive Class of Small RNAs in Caenorhabditis elegans,” Science 294, 862 (2001)

(11) S. M. Hammond et al., “Argonaute2, a Link Between Genetic and Biochemical Analyses of RNAi,” Science 293, 1146 (2001)

(12) E. Bernstein et al., “Role for a Bidentate Ribonuclease in the Initiation Step of RNA Interference,” Nature 409, 363 (2001)

(13) A. Fire et al., “Potent and Specific Genetic Interference by Double-Stranded RNA in Canorhabditis elegans,” Nature 391, 806 (1998)

(14) A. R. van der Krol et al., “Inhibition of Flower Pigmentation by Antisense CHS Genes: Promoter and Minimal Sequence Requirements for the Antisense Effect,” Plant Mol. Biol. 14, 457 (1990)

(15) C. Napoli et al., “Introduction of a Chimeric Chalcone Synthetase Gene in Petunia Results in Reversible Cosuppression of Homologous Genes in trans,” Plant Cell 2, 279 (1990)

What is claimed is:

1. A computer method for stopping the transcription of RNA that would otherwise be used to form a connectron comprising determining a total systematic control such that the first instance of a C1/C2 sequences to be expressed inhibits all other instances of the same C1/C2 sequences from being expressed.

2. A computer method that shows how the transcription of RNA that would otherwise be used to form a connectron can be stopped comprising determining a total systematic control such that the first instance of a C1/C2 sequences to be expressed inhibits all other instances of the same C1/C2 sequences from being expressed.
